(19) United States
(12) Patent Application Publication
(10) Pub. No.: US 2009/0204151 A1
(43) Pub. Date: Aug. 13, 2009

(54) Spinal implant device, procedure and system

(76) Inventor: Scott Bracken, Norfolk, VA (US)

Correspondence Address: Bradley D. Goldizen, HC 80 BOX 261, Maysville, WV, 26833, US

(21) Appl. No.: 12/322,757

(22) Filed: Feb. 6, 2009

Related U.S. Application Data
(60) Provisional application No. 61/063,975, filed on Feb. 7, 2008.

(51) Int. Cl. A61B 17/70

(52) U.S. Cl. 606/246; 606/276; 606/263

(57) ABSTRACT

A spinal implant procedure and system includes vertical rods fastened to the spine on opposite sides. A cross connector is arranged on each vertical rod and to opposite ends of a cross member. The cross member includes a block having a lateral opening and that includes a forked end that is arranged at a bottom side of a spineous process. A strap fastener include openings through which the cross member passes. An opening of the strap fastener hooks on a catch on the side of the block. The block limits prevents rotation, blocks extension and limits flexion of the affected vertebrae.

The present application relates to and claims priority from U.S. Provisional Application Ser. No. 61/063,975 filed on Feb. 7, 2008.

The present application was not subject to federal research and/or development funding.

### TECHNICAL FIELD

The invention generally relates to medical devices and procedures used in some spinal surgeries to immobilize certain vertebrae of the spine. More particularly, the invention relates to an improved medical procedure and spinal fixation system for use in medical procedures performed during spinal surgery. The surgical device is a block that couples to a spineous process to immobilize an injured region of the spine. A pair of fixed rods are affixed in bone screws and arranged on opposite sides of a spine. A cross member is arranged between the fixed rods and passes through the block that includes a notch. A strap, having a plurality of openings arranged therein, is wrapped about the block to secure it to the spineous process. The cross member passes through a through opening in the block and a pair of the openings provided in the strap. An opening in the second end of the strap passes over a hook arranged on one side of the block for seating the block against the spineous process.

### BACKGROUND OF THE INVENTION

Previously, spinal surgery has included fusing of two or more vertebrae together or implanting rods on either side of the spine to reinforce a damaged or deteriorated area of the spine. U.S. Pat. No. 7,306,060 to Janowski et al., incorporated by reference hereto, discloses a bone screw for use in attaching a rod to a bone of a patient, as shown in FIG. 5. U.S. Pat. No. 6,139,548 to Errico, incorporated by reference hereto, discloses a pair of rods implanted on either side of a damaged spine.

A major problem associated with fusing vertebrae and the prior art is the continued deterioration of vertebrae near the site of the spinal surgery. Deterioration of vertebra above and below the point of attachment of the prior art devices is believed to be caused by the misapplication of forces onto the point of attachment at the bone screws. The prior art fails to fully block the extension of the fused vertebrae and limit the flexion of them. Thus, the vertebrae in and around the affected area continue to experience deterioration.

In a healthy spine, the vertebrae are stacked upon one another and one's body weight is normal to the soles of the feet. Thus, the weight of the body is centrally located about the spine and is said to be “centerline loaded.” If forces and loads are experienced on the exterior regions of the vertebrae in an unnatural manner, the vertebrae will deteriorate over time. That is, while ordinarily being rugged and strong, a spine repeatedly receiving forces that are not centerline loaded, ie. having a direction normal or tangential to a vertebra, will degenerate over time. Such degeneration will not only deteriorate the vertebra but also the disks between the vertebra and cause pain and injury to the peripheral nerves.

In prior art devices forces applied to the spine are absorbed by rods on either side of the spine. A force normal in direction to either rod, or having a direction not parallel to the spine, is absorbed through the rods and transferred to healthy vertebrae on either side of the affect region through the bone screws. Such forces tend to create a moment of inertia about the bond screws. As the bone of the vertebrae does not exhibit such characteristics as are necessary to resists these moments of inertia, the bone deteriorates over time and quickly in certain instances. That is, since any force not center loaded tends to exacerbate the medical condition. The phenomenon is referred to in the medical industry as Adjacent Level Disease (ALD). If unequal forces are applied to the rods arranged on either side of the spine, then the patient may lean to one side and experience much pain and discomfort. Over time, activities like walking or setting causes more damage and deterioration to the affected area of the patient's spine. Thus, patients who have the prior art devices implanted may experience only temporary relief from pain and eventually require the replacement of the prior art device.

The current invention improves on these devices and procedures by adding a cross member that includes a block. This cross member promotes centerline loading and utilizes a spineous process of a health vertebra to resist moments of inertia about the bone screws. The block includes a forked end and solidly attaches to the spineous process via a strap fastener. Each end of the cross member attaches to a vertical rod via a cross connector.

### SUMMARY OF THE INVENTION

The present invention is a spinal implant block device and system that improves on the prior art. The system includes a block having a forked end with two tines. The tines are arranged on opposite sides at the base of the spineous process. The base of the forked end is preferably arranged in a notch on a bottom side at the base of the spineous process.

The block includes a lateral opening through which the cross member passes to solidly fasten it to the spineous process. A strap fastener includes a plurality of openings. Two of such openings in the strap fastener are arranged next to or substantially near opposite ends of the lateral opening. A different one of the openings in the strap fastener is used to secure the block to the spineous process via a catch or hook. The catch or hook is preferably provided on the same side as the end of the lateral opening into which the connecting rod first passes.

The cross member is inserted through a first opening in the plurality openings in the strap fastener before it is passed into and enters the lateral opening. A second opening of the plurality of openings is arranged next to the second end of the lateral opening through which the cross member exits. That is, the strap is passed across the bottom edge of the block with the cross member being passed first through one of the openings in the strap fastener and thereafter into the first end of the lateral opening. After exiting the lateral opening on an opposite side of the block, the cross member then passes through a second opening in the strap fastener.

In this manner, the strap fastener is wrapped around a bottom edge of the block and secured in place. The strap fastener then extends upward along a first side of the block, across an upper region of the base of the spineous process, and downward along a second side of the block opposite the first side to have a third opening from the plurality of openings be hooked onto a catch fixed on the second side of the block.

Thus, the strap fastener is wrapped about the spineous process to couple the block to the spineous process such that a vertebra above or below a damaged or deteriorated region of the spine may be reinforced or stabilized to aid in the recovery of a patient after spinal surgery. The process and system stabilize vertical rods fastened on either side of the spine and between which the cross member is fastened. For purposes of the disclosure, the terms “vertical rod” and “vertical rods” refers to any rod arranged on either side of the spine and arranged substantially vertical when the patient is standing. Moreover, any reference with respect to upper and lower should be considered interchangeable if the system is orientated in a reverse direction. That is, the block may be provided above or below an injured region of the spine. Likewise, the skilled artisan can recognize that a plurality of the cross members may be provided to immobilize an area of the spine.

The cross member is fastened between the vertical rods via a pair of cross connectors, as shown in FIGS. 3A-B and FIGS. 4A-C. A cross connector is arranged at each end of the cross member. Each cross-connector includes a pair of apertures. In the first embodiment as shown in FIGS. 3A-B, the first aperture is a ring aperture through which a respective vertical rod pass. An end of the cross member is fastened into the second aperture to couple the cross member to the vertical rod. In a second embodiment, the cross connector includes a recess for accepting the cross member and a first set screw secures an end of the cross member within the cross connector. A curved end extends from the bottom of the cross connector. A second set screw and the arcuate surface of the curved end defines a recess through which the vertical rod passes. As can be understood by those skilled in the art, tightening the second set screw causes the rod to be pressed against the curved surface to seize the vertical rod to prevent the cross connector from slip along the vertical rod. It should be recognized that certain modification to cross connector may be made such that the cross member and vertical rod may be reversed as necessary.

In the manner of the first embodiment, the vertical rods pass through the ring of the cross connectors before the rods attach to the bone screw construct. The tulip head on the cross connector accepts the horizontal rod that passes through the extension block. The tulip head may include a retaining ring, clip, wire, cam or other type of fastening means. In this manner, the implant device prevents rotation, blocks extension and limits flexion of the affected vertebrae. Otherwise the vertical rods may pass between a set screw and a curved surface as shown in FIGS. 4A-4C of the second embodiment.

The various parts of the invention including the vertical rods, cross connectors, cross members and bone screws are preferably formed from a hard metal or composite material which is rigid, inert and hypoallergenic. Titanium is the preferred material for use in implementing the invention. The preferred diameter of the cross member and vertical rods is 5.5 mm. The cross connectors are preferably 10 mm in diameter at their largest point. The device is added to any spinal fusion operation to prevent or inhibit ALD.

It is an object of the invention to improve on the prior art and provide a superior system that prevents the deterioration of vertebrae on either side of a damaged region of the spine that is repaired by having rods fastened on either side of the damaged region.

It is a further object of the invention to provide a system and procedure that will improve on the quality of life for patients having spinal surgery.

Additional objects and advantages of the invention will be set forth in part in the description which follows, and in part will be obvious from the description, or may be learned from practicing the invention. The objects and advantages of the invention will be obtained by means of instrumentalities in combinations particularly pointed out in the appended claims.

### BRIEF DESCRIPTION OF THE DRAWINGS

FIG. 1 is a view of the improved surgical device and system.

FIG. 2A is a plan view of the spinal block when viewed from a back side. FIG. 2B is a perspective view of the spinal block.

FIG. 3A is a cross section view of a cross connector of the first embodiment. FIG. 3B is an overhead plan view of a cross connector shown in FIG. 3A.

FIG. 4A is a side plan view of a cross connector of the second embodiment whit a vertical reinforcing rod taken in cross section. FIG. 4B is an overhead plan view the cross connector of the second embodiment. FIG. 4C is a side plan view of the cross connector of the second embodiment with the cross member taken in cross section.

FIG. 5 shows the strap fastener that fastens the block to the spineous process.

FIG. 6A is an exploded prior art bone screw assembly for use with the invention. FIG. 6B shows the prior art bone assembly configured for use in securing vertical rods.

### DETAILED DESCRIPTION OF THE INVENTION

The embodiments of the invention and the various features and advantageous details thereof are more fully explained with reference to the non-limiting embodiments and examples that are described and/or illustrated in the accompanying drawings and set forth in the following description. It should be noted that the features illustrated in the drawings are not necessarily drawn to scale, and the features of one embodiment may be employed with the other embodiments as the skilled artisan recognizes, even if not explicitly stated herein. Descriptions of well-known components and techniques may be omitted to avoid obscuring the invention. The examples used herein are intended merely to facilitate an understanding of ways in which the invention may be practiced and to further enable those skilled in the art to practice the invention. Accordingly, the examples and embodiments set forth herein should not be construed as limiting the scope of the invention, which is defined by the appended claims. Moreover, it is noted that like reference numerals represent similar parts throughout the several views of the drawings.

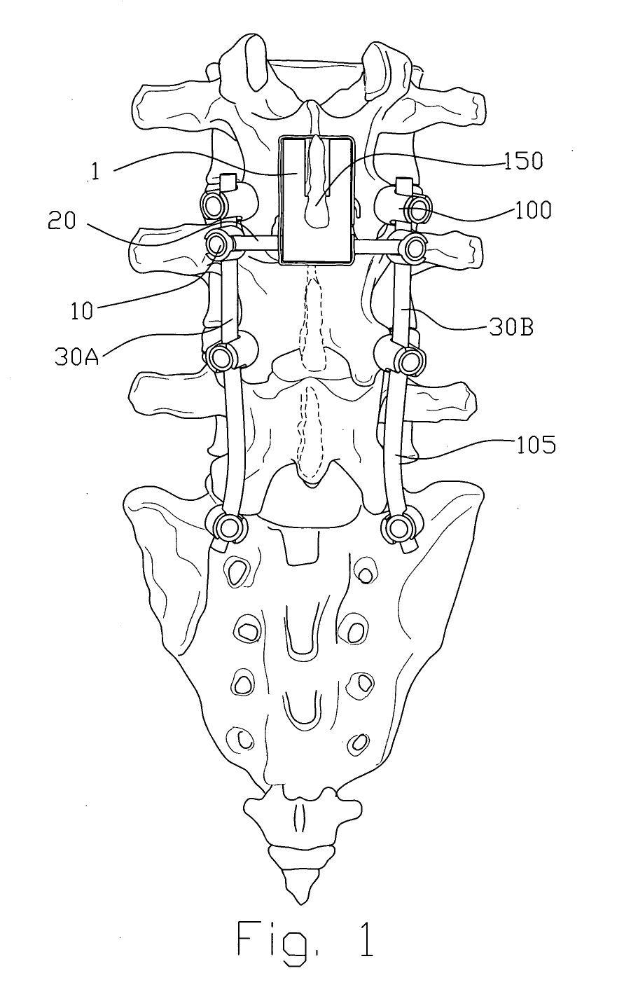

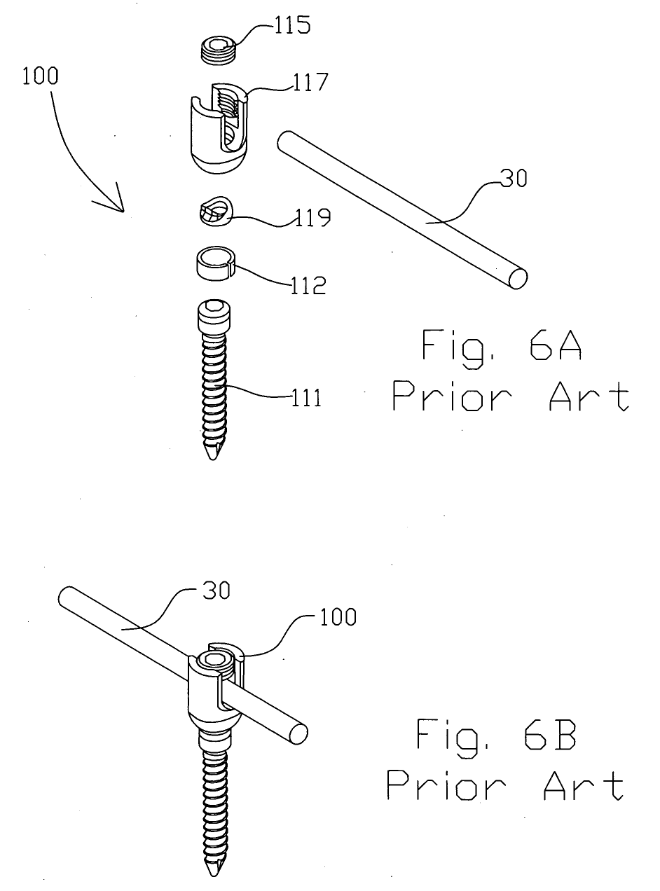

As shown in FIG. 1, the system, device and process include using bone screws 100 to secure two vertical rods 30A, 30B on opposite sides of an injured spine that includes fused vertebrae. The bones screws 100 and vertical rods 30A-B may be of those types mentioned in the prior art references above. As shown in FIG. 6A-B, one such bone screw 100 includes a threaded base 111 which is preferably drilled into a bony portion of the spine such as the pedicle portion of a vertebra. Such bone screw is shown and disclosed in U.S. Pat. No. 7,306,606 to Sasing. The bone screw 100 further includes a spherical retaining ring 112, a spacer 119, a coupling member 117 and a setscrew 115. Rod 30 is arranged within coupling member 117 via setscrew 115.

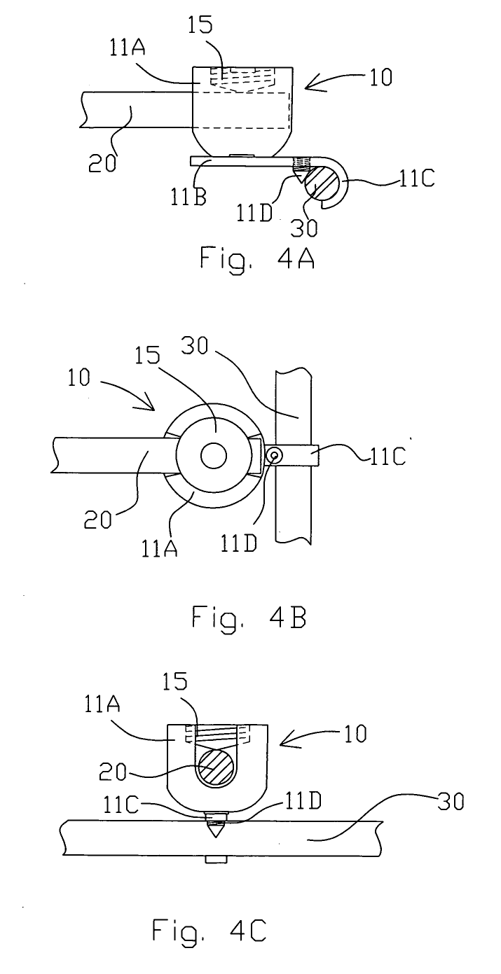

A pair of cross connectors 10 are arranged at opposite ends of the cross member 20 and couple it to the vertical rods 30A-B,. As shown and can be understood by those skilled in the art, a block 1 is arranged on the cross member 20 between the two vertical rods 30A-B. The cross member 20 extends through a lateral opening 5 in the block 1 as can be more clearly understood when viewing FIGS. 3A-4C.

A forked end 1B of the block is arranged at the base of the spineous process and a strap fastener 40 is wrapped around the block 1 and the spineous process to couple the block 1 to the spineous process. The strap fastener 40 includes a plurality of openings comprising openings 41, 42, 43. A first opening 41 near an end of the strap fastener 40 is arranged next to one end of the lateral opening 5 and the cross member is passed through this first opening and into the lateral opening. The cross member 20 extends through the lateral opening 5 and out the other side of the block. Thereafter, the cross member 20 extends through a second opening 42 in the strap 40 and the strap is wrapped around the side of the block and across the ends of the tines 2A-B and the top edge of the base of the spineous process. A third opening 43 in the strap fastener 40 is fastened onto a hook 4 on the side of the block that includes the end into which the cross member was first passed as shown in FIG. 1.

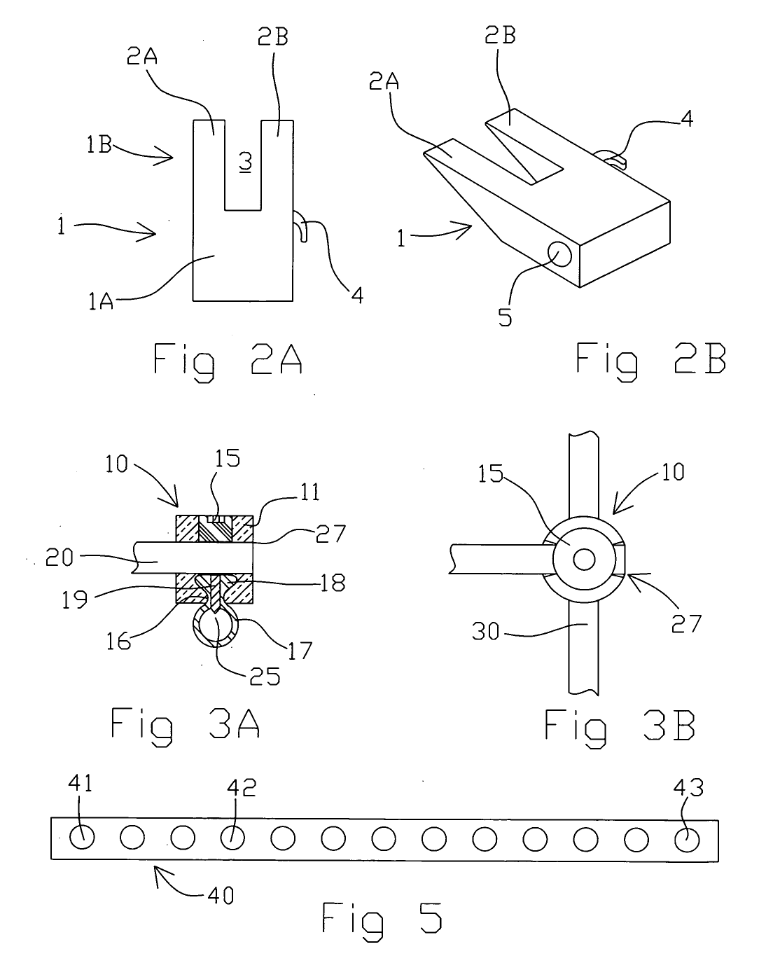

FIGS. 2A and 2B show the block 1. The block 1 includes a forked end 1A that comprises two tines 2A-B. The tines 2A-B extend from an upper edge of the block to be arranged on opposite sides of the spineous process. A lateral opening extends through the block near a bottom edge 1A. A catch 4 is formed along a side of block. An overall length of the block may vary according the size of the patient. The preferred length of a tine is 20 mm. The overall lateral width of the block is preferably 22 mm. The diameter of the lateral opening is preferably 5.5 mm. The catch 4 is preferably 2 mm in thickness. As can be understood by FIG. 2B one side of the tines 2A-B may be sloped.

FIG. 3A shows a cross section view of a first embodiment of the cross connector 10. The cross connector 10 includes an orifice 27 that accommodates an end of cross member 20. A pressure exerting device such as setscrew 15 is arranged in the upper center of the cross connector 10 and mates with internal threads within the cross connector 10 to exert pressure onto the cross member 20 to secure it to the cross connector 10. The cross member 20 extends perpendicular to the vertical rods 30A-B, one of which is fastened within the second opening 25 of the cross connector 10. The second opening 25 is defined by a lower ring 17 having an upper end 18 secured within an orifice 16 and secured by a setscrew 19 which forces the upper end 18 outward. Otherwise, either rod 20, 30A-B may be fastened to the cross connector s10 via a tulip end. The tulip end may include a retaining ring, clip, wire or cam for fastening the cross connecctor to the cross member. That is the tulip end is a type of twist fastener that quickly couples the cross connector to the cross member with a twist that is preferably ninety degrees of rotation instead of threading a setscrew into the cross connector. The first opening is preferably 5.5 mm in diameter. The second opening in the cross connector includes rounded lower edges and is preferably 5.5 mm in diameter. If used, the tulip end is preferably 8 mm in width. The overall width of the block is preferably 13 mm in width. The height of the cross connector is 14 mm not included the ring opening.

FIGS. 4A-4C depict a second embodiment of the cross connector 10. As can be understood, the cross connector 10 includes a body 11A that comprises threads for accepting setscrew 15. Otherwise, a tulip connector, as shown and disclosed in U.S. Patent Application Publication US 2006/0206114 A1 which is incorporated by reference hereto, may be used to exert pressure to hold either the vertical rod or cross member in place. The body 11A is preferably cup-shaped and includes a lower extension 11B that comprises a curved end 11C that accepts on of either the cross member or the vertical rod. A second setscrew 11D preferably secures vertical rod 30 into place as shown.

FIG. 5 show a strap fastener that includes a plurality of openings therein. The openings are used as discussed above for passing the cross member there through and fastening a second end of the strap fastener to the hook on the side of the block. FIG. 6 shows a bone screw assembly and vertical rod for use with the invention. The vertical rod is arranged within the bone screw assembly and fastened thereto.

It is to be understood that the invention is not limited to the exact construction illustrated and described above, but that various changes and modifications may be made without departing from the spirit and the scope of the invention as defined in the following claims. While the invention has been described with respect to preferred embodiments, it is intended that all matter contained in the above description or shown in the accompanying drawings shall be interpreted as illustrative and not in limiting sense. From the above disclosure of the general principles of the present invention and the preceding detailed description, those skilled in the art will readily comprehend the various modifications to which the present invention is susceptible. Therefore, the scope of the invention should be limited only by the following claims and equivalents thereof.

I claim:

1. A system for repairing a deteriorated or injured region of the spine, said system including:
a first plurality of bone screws fastened into vertebra on the left side of the spine, each bone screw having an opening accessible by a pressure fastener;
a second plurality of bone screws fastened into vertebra on the right side of the spine, each bone screw having an opening accessible by a pressure fastener;
a first rod passing through the opening in each bone screw in the first plurality of openings to create a rigid skeleton on the left side of the spine to span across a region of at least one deteriorated vertebra;
a second rod passing through the opening in each bone screw in the first plurality of openings to create a rigid skeleton on the right side of the spine to span across a region of at least one deteriorated vertebra;
a cross member having a first end and a second end, said first end coupled to the first rod, said second end coupled to the second rod;
a first cross connector being arranged at the first end of the cross member and coupling the cross member to the first rod;
a second cross connector being arranged at the second end of the cross member and coupling the cross member to the second rod; and,
a block having a first end comprising a pair of forks engaging a spineous process of a vertebra such that a notch defined by the forks accepts a lower region of the spineous process, a lateral opening extending through a second end of the block and receiving the cross member there through.

2. The system for repairing a deteriorated or injured region of the spine of claim 1 further comprising:
a block that includes a hook extending from one side thereof, said hook arranged parallel to a plane extending from the first rod to the second rod; and,
a strap having a first end and a second end and a plurality of openings spaced apart from one another there between, the cross member passing through one of the openings of the plurality of openings, said strap being wrapped about the spineous process and an opening of the strap substantially near the second end passes across the hook of the block to couple the block and spineous process together.

3. The system for repairing a deteriorated or injured region of the spine of claim 2 wherein the hook is preferably 2 mm in thickness.

4. The system for repairing a deteriorated or injured region of the spine of claim 1 wherein at least the cross member and vertical rods comprise titanium.

5. The system for repairing a deteriorated or injured region of the spine of claim 1 further comprising wherein a diameter of the cross member and the first and second vertical rods is 5.5 mm

6. The system for repairing a deteriorated or injured region of the spine of claim 1 further comprising wherein a diameter of the cross connectors when taken in plan from above is 10 mm.

7. The system for repairing a deteriorated or injured region of the spine of claim 1 further comprising a block having a tine which is 20 mm in length.

8. The system for repairing a deteriorated or injured region of the spine of claim 1 further comprising a block having an overall lateral width of 22 mm.

9. The system for repairing a deteriorated or injured region of the spine of claim 1 further comprising a block having a lateral opening which is preferably 5.5 mm in diameter.

10. The system for repairing a deteriorated or injured region of the spine of claim 1 wherein a base of the forked end is preferably arranged in a notch on a bottom side at the base of the spineous process.

11. The system for repairing a deteriorated or injured region of the spine of claim 1 wherein the cross connector comprises a tulip end that includes one or more selected from a group consisting of a retaining ring, clip, wire or cam for fastening the cross connecctor to the cross member.

12. The cross connector of claim 11 wherein a diameter of the tulip end is 8 mm.

13. The system for repairing a deteriorated or injured region of the spine of claim 1 wherein an overall width of the block is 13 mm.

14. The system for repairing a deteriorated or injured region of the spine of claim 1 wherein the height of the cross connector is 14 mm excluding the ring opening.

15. A medical system for repairing a damaged spine, said system comprising:
a first plurality of bone screws fastened into vertebrae on the left side of the spine, each bone screw having an opening accessible by a threaded fastener;
a second plurality of bone screws fastened into vertebrae on the right side of the spine, each bone screw having an opening accessible by a threaded fastener;
a first rod passing through the opening in each bone screw of the first plurality of bone screws to create a rigid skeleton on the left side of the spine to span across a region of at least one deteriorated vertebra;
a second rod passing through the opening in each bone screw of the second plurality of bone screws to create a rigid skeleton on the right side of the spine to span across a region of the at least one deteriorated vertebra;
a cross member having a first end and a second end, said first end coupled to the first rod, said second end coupled to the second rod;
a first cross connector arranged at the first end of the cross member and coupling the cross member to the first rod;
a second cross connector arranged at the second end of the cross member and coupling the cross member to the second rod;
a block having top edge comprising a forked end with two tines and a bottom edge at the opposite end, said tines being arranged on opposite sides at a base of a spineous process of a vertebra in the damaged spine, said block further comprising a first side and a second side, said block further includes a lateral opening with a first end and a second end and through which the cross member passes to solidly fasten the block to said spineous process such that said lateral opening extends from the first side to the second side of the block; a catch extending from the block; and, p1 a strap fastener comprising a plurality of openings, two openings of the plurality of openings in the strap fastener are arranged in substantial proximity to the first and second ends of the lateral opening, wherein said cross member is inserted through one of the two openings arranged in substantial proximity to the first end of the lateral opening before entering the first end of the lateral opening in the block, the other opening of the two such openings of the plurality of openings is arranged next to the end of the second end of the lateral opening through which the cross member exits, such that the strap fastener is wrapped around the bottom edge of the block, said strap fastener extending upward along a first side of the block, across the base of the spineous process, and downward along a second side of the block opposite the first side to have a third opening from the plurality of openings be hooked onto a catch fixed on the second side of the block wherein said strap fastener is wrapped about the block and the spineous process to couple the block to the spineous process such that a vertebra above or below a damaged or deteriorated region of the spine may be reinforced and stabilized to aid in the recovery of a patient after spinal surgery.

16. The medical system of claim 15 wherein a base of the forked end of the block is preferably arranged in a notch on a bottom side at the base of the spineous process.

17. The medical system of claim 15 wherein at least the cross member and vertical rods comprise titanium.

18. The medical system of claim 15 wherein a diameter of the cross member and the vertical rods is 5.5 mm.

19. A process for repairing an injured or deteriorated spine, comprising:
fastening a first plurality of bone screws into vertebrae on the left side of the spine, each bone screw having an opening accessible by a threaded fastener;
fastening a second plurality of bone screws into vertebrae on the left side of the spine, each bone screw having an opening accessible by a threaded fastener;
passing a first rod through the opening in each bone screw of the first plurality of bone screws to create a rigid skeleton on the left side of the spine to span across a region of at least one deteriorated vertebra;
passing a second rod through the opening in each bone screw of the second plurality of bone screws to create a rigid skeleton on the right side of the spine to span across a region of at least one deteriorated vertebra;
fastening a cross member having a first end and a second end such that said first end is coupled to the first rod and said second end is coupled to the second rod such that a first cross connector is arranged at the first end of the cross member and coupling the cross member to the first rod and a second cross connector is arranged at the second end of the cross member and coupling the cross member to the second rod;
arranging a block onto the cross member, said block having a top edge comprising a forked end with two tines and a bottom edge at the opposite end, said tines being arranged on opposite sides at a base of a spineous process of a vertebra in the damaged spine, said block further comprising a first side and a second side, said block further includes a lateral opening with a first end and a second end and through which the cross member passes to solidly fasten the block to said spineous process such that said lateral opening extends from the first side to the second side of the block; a catch extending from the block; and,
wrapping and fastening a strap fastener about the block to secure the block to the spineous process, said strap fastener comprising a plurality of openings, two openings of the plurality of openings in the strap fastener are arranged in substantial proximity to the first and second ends of the lateral opening, wherein said cross member is inserted through one of the two openings arranged in substantial proximity to the first end of the lateral opening before entering the first end of the lateral opening in the block, the other opening of the two such openings of the plurality of openings is arranged next to the end of the second end of the lateral opening through which the cross member exits, such that the strap fastener is wrapped around the bottom edge of the block, said strap fastener extending upward along a first side of the block, across the base of the spineous process, and downward along a second side of the block opposite the first side to have a third opening from the plurality of openings be hooked onto a catch fixed on the second side of the block wherein said strap fastener is wrapped about the block and the spineous process to couple the block to the spineous process such that a vertebra above or below a damaged or deteriorated region of the spine may be reinforced and stabilized to aid in the recovery of a patient after spinal surgery.
